(19) United States
(12) Patent Application Publication
(10) Pub. No.: US 2009/0246833 A1
(43) Pub. Date: Oct. 1, 2009

(54) Small interfering RNAS as non-specific drugs

(75) Inventors: Joao Trindade Marques, Evanston, IL (US); Bryan R. G. Williams, Kallista (AU)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Cleveland Clinic Foundation, Cleveland, OH (US)

(21) Appl. No.: 12/079,594

(22) Filed: Mar. 27, 2008

Related U.S. Application Data
(63) Continuation of application No. PCT/US2006/038542, filed on Sep. 29, 2006.
(60) Provisional application No. 60/722,649, filed on Sep. 29, 2005.

(51) Int. Cl. C12P 19/34; C12N 5/00

(52) U.S. Cl. 435/91.1; 435/375

(57) ABSTRACT

The present invention is directed to a method of modulating (e.g., inducing, inhibiting) activation of a double stranded RNA (dsRNA) signaling pathway, such as the dsRNA signaling pathway that accompanies RNA interference (RNAi) of a target RNA sequence, in a cell, comprising introducing into the cell small interfering RNA (siRNA) that degrades the target RNA sequence, and maintaining the cell under conditions in which RNAi of the target RNA sequence occurs and activation of the dsRNA signaling pathway is modulated in the cell.

### RELATED APPLICATIONS

This application is a continuation of International Application No. PCT/US2006/038542, which designated the United States and was filed on 29 Sep. 2006, published in English, which claims the benefit of U.S. Provisional Application No. 60/722,649, filed on Sep. 29, 2005. The entire teachings of the above applications are incorporated herein by reference.

### GOVERNMENT SUPPORT

The invention was supported, in whole or in part, by grants RO1 AI 34039 and PO1 CA 62220 from National Institute of Health (NIH). The Government has certain rights in the invention.

### BACKGROUND OF THE INVENTION

RNA interference (RNAi) is a mechanism of post-transcriptional gene silencing directed by double stranded RNA (dsRNA) (Meister G, Tuschl T., Nature. 431, 343-9, (2004)). Exogenous dsRNA molecules introduced into cells are processed by the RNase III enzyme Dicer into duplexes of 21-25 nucleotides (nt) containing 5′ monophosphates and 2-nt 3′ overhangs referred to as small interfering RNAs (siRNAs) (Bernstein, E., et al., Nature. 409, 363-6 (2001); Elbashir, S. M., et al., Genes Dev. 15, 188-200 (2001)). The siRNAs are incorporated into a multi-protein RNA-induced silencing complex (RISC) that degrades RNAs with sequences complementary to the siRNA (Tomari, Y., Zamore, P. D., Genes Dev. 19, 517-29 (2005)). Endogenous micro RNAs (miRNAs) also use the RNAi machinery to regulate gene expression (Ambros, V., Nature. 431, 350-5 (2004)). miRNAs are transcribed by RNA polymerase II as long precursors that are processed in the nucleus by the RNase III enzyme Drosha into ˜65 nt short hairpin RNAs (shRNAs) containing 2-nt 3′ overhangs (Cullen, B. R., et al., Mol. Cell, 16:861-865 (2004)). Exportin 5 then exports the premiRNAs to the cytoplasm where they are processed by Dicer into mature miRNAs with 2-nt 3′ overhangs (Kim, V. N., Nat Rev Mol Cell Biol. 6, 376-85 (2005)).

dsRNA is a common intermediate of viral replication that activates signaling pathways involved in mammalian antiviral defense (Williams, B. R., Sci STKE. 89:RE2 (2001)). These intracellular dsRNA signaling pathways are present in most mammalian cell types with possibly the exception of undifferentiated cells (Chen, W., et al., FEBS Lett. 579, 2267-72 (2005); Yang, S., et al., Mol Cell Biol. 21, 7807-16 (2001)). The activation of dsRNA signaling pathways, in contrast to RNAi, can induce sequence independent protein synthesis inhibition and RNA degradation (Marques, J. T. et al., J. Virol. 79, 11105-14 (2005)). This is carried out by the dsRNA-activated protein kinase (PKR) and 2′5′ Oligoadenylate synthetase (OAS)/RNase L pathways, both which are activated directly by dsRNA (Williams, B. R., Sci STKE. 89:RE2 (2001); Samuel, C. E., Clin Microbiol Rev. 14, 778-809 (2001)). In addition, dsRNA induces the expression of antiviral genes (Sarkar, S. N., et al., Pharmacol Ther. 103, 245-59 (2004)) including early genes that are directly regulated by the transcription factors IRF-3 and NF-KB and late genes whose expression requires type I Interferons (IFNs) produced in the early phase of viral infection (Marie, I., et al., EMBO J. 17, 6660-9 (1998)); Hata, N. et al., Biochem Biophys Res Commun. 285, 518-25 (2001); Nakaya, T. et al., Biochem Biophys Res Commun. 283, 1150-6 (2001); Elco, C. P., et al., J Virol. 79, 3920-9 (2005)). RIG-I and Mda-5, two IFN-inducible RNA helicases containing caspase recruitment domains (CARD), along with PKR, can mediate the activation of IRF-3 and NF-KB in response to intracellular dsRNA (Williams, B. R., Sci STKE. 89:RE2 (2001); Diebold, S. S. et al., Nature. 424, 324-8 (2003); Yoneyama, M. et al., Nat Immunol. 5, 730-7 (2004); Andrejeva, J. et al., Proc Natl Acad Sci USA. 101, 17264-9 (2004)). Diverse cell types derived from RIG-I knock out mice have impaired responses to viral dsRNA establishing the essential role of RIG-I in the mammalian antiviral response (Kato, H. et al., Immunity. 23, 19-28 (2005)). Interestingly, in invertebrates and plants, RNAi serves as a powerful antiviral defense and recent evidence suggests that mammalian cells can also use RNAi as an antiviral mechanism (Voinnet, O., Nat Rev Genet. 6, 206-20 (2005); Bennasser, Y., et al., Immunity. 22, 607-19 (2005)). It remains unclear whether the RNAi and dsRNA signaling pathways interact to maximize antiviral defense in mammals.

RNAi is emerging as a potent tool to regulate gene expression in experimental and clinical settings (Hannon, G. J., Rossi, J. J., Nature. 431, 371-8 (2004)). Consequently, it is essential that any potential nonspecific effects be minimized. For example, in mammalian cells, siRNAs have been shown to activate IFN production as a side effect (Sledz, C. A., et al., Nat Cell Biol. 5, 834-9 (2003); Kim, D. H. et al., Nat Biotechnol. 22, 321-5 (2004); Persengiev, S. P., et al., RNA. 10, 12-8 (2004)), but the mechanisms involved remain to be determined. Furthermore, the existence of self dsRNAs such as miRNAs raises questions about how these endogenous molecules avoid recognition by the antiviral pathways. Therefore an understanding of how mammalian cells discriminate self from non-self RNAs would aid in improving the specificity of siRNAs and their usefulness as therapeutic tools.

### SUMMARY OF THE INVENTION

The activation of mammalian antiviral systems by small interfering RNAs (siRNAs) complicates the use of RNA interference (RNAi) to specifically down-regulate gene expression. As described herein, to uncover the basis of these non-specific effects, the effect of chemically synthesized siRNAs on mammalian double-stranded RNA (dsRNA) activated signaling pathways was analyzed. siRNAs as short as 21 nucleotides were potent activators of IRF3-mediated gene induction as long as they lacked the 3′ overhangs characteristic of Dicer products. The 3′ overhangs impair the ability of the RNA helicase RIG-I to unwind the dsRNA substrate and activate downstream signaling to IRF3. These findings provide a basis for rational design of siRNAs capable of modulating the activation of antiviral pathways. This allows for direct targeting of genes without non-specific effects or, alternatively, for induction of bystander effects to potentially increase the efficacy of siRNA-based treatments of viral infections or cancer.

Accordingly, the present invention is directed to a method of modulating (e.g. inducing, inhibiting) activation of a double stranded RNA (dsRNA) signaling pathway, such as the dsRNA signaling pathway that accompanies RNA interference (RNAi) of a (one or more) target RNA sequence, in a (one or more) cell, comprising introducing into the cell small interfering RNA (siRNA) that degrades the target RNA sequence, and maintaining the cell under conditions in which RNAi of the target RNA sequence occurs and activation of the dsRNA signaling pathway is modulated in the cell.

In one embodiment, the invention is directed to a method of inducing activation of a double stranded RNA (dsRNA) signaling pathway that accompanies RNA interference (RNAi) of a target RNA sequence in a cell. In this embodiment of the invention, the siRNA that is introduced into the cell can be double stranded and can comprise at least one blunt end. For example, the siRNA can be an siRNA wherein both ends are blunt-ended; an siRNA wherein one end is blunt-ended and the other end comprise a 5′ 2 nucleotide overhang; an siRNA wherein one end is blunt-ended and the other end comprises a 3′ 2 nucleotide overhang; and/or a combination thereof. Alternatively, in this embodiment, the siRNA that is introduced into the cell can be double stranded and comprise a 5′ 2 nucleotide overhang at each end. In a particular embodiment, an overhang can comprise from about 1 nucleotide to about 5 nucleotides.

In another embodiment, the invention is directed to a method of inhibiting activation of a double stranded RNA (dsRNA) signaling pathway that accompanies RNA interference (RNAi) of a target RNA sequence in a cell. In this embodiment of the invention, the siRNA that is introduced into the cell can be double stranded and comprise at least 2 overhangs. An overhang can comprise from about 1 nucleotide to about 5 nucleotides. In a particular embodiment, each of the at least 2 overhangs comprise 2 nucleotides. In this embodiment of the invention, the siRNA can be an siRNA wherein both 3′ ends comprise a 2 nucleotide overhang; an siRNA wherein one end comprises a 3′ 2 nucleotide overhang and the other end comprises a 5′ 2 nucleotide overhang; and/or a combination thereof.

The invention is also directed to a method of degrading a target RNA sequence using RNA interference (RNAi) in the absence of non-specific effects in a cell, comprising introducing into the cell small interfering RNA (siRNA) that degrades the target RNA sequence, wherein the siRNA is double stranded and comprises at least 2 overhangs, and maintaining the cell under conditions in which the target RNA sequence is degraded by the siRNA in the absence of non-specific effects.

Also encompassed by the present invention is a method of degrading a target RNA sequence using RNA interference (RNAi) in the presence of non-specific effects in a cell, comprising introducing into the cell small interfering RNA (siRNA) that degrades the target RNA sequence, wherein the siRNA is double stranded and comprises at least one blunt end, and maintaining the cell under conditions in which the target RNA sequence is degraded by the siRNA in the presence of non-specific effects. In a particular embodiment, the non-specific effect is activation of a double stranded RNA (dsRNA) signaling pathway which results in an inflammatory response and/or apoptosis in the cell.

The present invention is also directed to a method of enhancing an antiviral effect induced using RNA interference (RNAi) in a cell, comprising introducing into the cell small interfering RNA (siRNA) that degrades a target RNA sequence of a virus (e.g., wherein the siRNA is double stranded and comprises at least one blunt end) and maintaining the cell under conditions in which the target RNA sequence of the virus is degraded by the siRNA in the presence of non-specific effects in the cell (e.g., promotes dsRNA signaling).

The invention also pertains to a method of enhancing an anticancer effect induced using RNA interference (RNAi) in a cell, comprising introducing into the cell small interfering RNA (siRNA) that degrades a target RNA sequence of a cancer (e.g., wherein the siRNA is double stranded and comprises at least one blunt end) and maintaining the cell under conditions in which the target RNA sequence of the cancer is degraded by the siRNA in the presence of non-specific effects in the cell (e.g., promotes dsRNA signaling).

In the methods of the present invention, the siRNA can comprise a sequence that is from about 19 nucleotides to about 30 nucleotides. In one embodiment, the siRNA can comprise a sequence that is from about 25 nucleotides to about 27 nucleotides. In a particular embodiment, the siRNA can comprises a sequence that is 27 nucleotides.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1a-1c show analysis of the activation of dsRNA signaling and RNAi by siRNAs. (FIG. 1a) siRNAs ranging from 23 to 27 nt in length that do not contain 3′ overhangs strongly activate dsRNA signaling pathways. The siRNAs at 80 mM concentration were transfected into T98G cells and after 48 h total cell extracts were prepared and analyzed by Western Blot for the expression of the dsRNA-induced protein 56K (p56). GFP silencing was by measuring fluorescence from the same lysates. A T7 RNA polymerase synthesized RNA (TAR) was used as a positive control for p56 induction. (FIG. 1b) The activation of dsRNA signaling is sequence independent, does not require expression of the mRNA target and is weakly triggered by dsDNA oligos. (FIG. 1c) Activation of dsRNA signaling pathways and RNAi show a concentration response. The western blots are representative of at least three independent experiments and the fluorescence graphs are an average of two independent readings.

FIGS. 2a-2b show expression of dsRNA-induced genes in response to siRNAs is delayed but does not require de novo protein synthesis. T98G were transfected with 80 nM of siRNA for the indicated times in the presence or absence of 5 μg/mL of cycloheximide to block de novo protein synthesis. (FIG. 2a) Total protein extracts were prepared and analyzed by Western Blot and (FIG. 2b) total RNA was extracted and the mRNA expression was analyzed by Real Time RT-PCR. The western blot is representative of two independent experiments and the real time PCR values are an average of two independent reactions.

FIGS. 3a-3f show cell type differences in the responses to chemically synthesized siRNAs. Comparison of p56 induction and GFP silencing by the siRNAs in T98G (FIG. 3a) and Hela cells (FIG. 3b). Induction of p56 by the 27+0 siRNA in HT1080 cells primed with IFN (FIG. 3c). (FIG. 3d) Over-expression of the IFN-inducible RNA helicase RIG-I restores dsRNA signaling in 293T cells in response to poly(I:C). (FIG. 3e) Activation of dsRNA signaling in response to chemically synthesized siRNAs 293T cells overexpressing the IFN-inducible RNA helicase RIG-I. (FIG. 3f) mRNA levels of the RNA helicase RIG-I in HT1080 cells after IFN treatment. The western blots are representative of four (FIG. 3a and FIG. 3b), two (FIG. 3c) and three (FIG. 3d and FIG. 3e) independent experiments and the real time PCR values (FIG. 3f) are an average of two independent reactions.

FIGS. 4a-4d show in vitro analysis of the interaction between the RNA helicase RIG-I and chemically synthesized siRNAs with or without 2-nt 3′ overhangs. (FIG. 4a) In vitro binding and unwinding assay of a 27+0 or a 27+2 siRNA in the presence of increasing concentrations of the Helicase domain of RIG-I. (FIG. 4b) Quantification of the dsRNA, ssRNA and the RNA/Protein complex formed in the presence of increasing amounts of the helicase domain of RIG-I with the 27+0 or the 27+2 siRNA. (FIG. 4c) ATPase activity of full-length RIG-I alone or in the presence of a 27+0 or a 27+2 siRNA. (FIG. 4d) Model for the differential recognition of dsRNAs with or without 3′ by the RNA helicase RIG-I. The results are representative of three (FIG. 4a) and two (FIG. 4c) independent experiments.

FIGS. 5a-5c show the importance of the ends of the siRNA for the activation of dsRNA signaling and RNAi. (FIG. 5a) Chemically synthesized siRNAs containing blunt ends or 5′ overhangs are more potent at activating dsRNA signaling than siRNAs containing 3′ overhangs or DNA ends. All duplexes were transfected at 75 nM into T98G, total cell extracts were prepared at 72 h and induction of p56 assessed by Western blot. (FIG. 5b) RNAi is triggered by all duplexes tested despite the overhangs or DNA modifications. Specific GFP silencing was also determined by measuring total fluorescence in the lysates. (FIG. 5c) Toxicity associated with each of the duplexes was determined 5 days after transfection at the indicated concentrations. The western blot is representative of three independent experiments and the fluorescence graph is an average of two independent readings. The toxicity results represent the average of biological triplicates with standard deviations.

FIGS. 6a-6c show the analysis of the activation of dsRNA signaling and RNAi by siRNAs. (FIG. 6a) Chloroquine, an inhibitor of endosomal acidification, does not inhibit p56 induction by the 27+0 siRNA nor T7 synthesized RNAs. Induction of p56 and silencing of GFP were analyzed in T98G cells 48 h after transfection with the 27+0 siRNA in serum-free, or media containing synthetic serum (FC3) or FBS (FIG. 6b) or incubated with the siRNA for different times (FIG. 6c). The results are representative of two independent experiments and the fluorescence graphs are an average of two independent readings.

FIG. 7 shows different kinetics of p56 induction in response to Poly(I:C) in T98G cells and 293T cells transfected with Flag-RIG-I. RIG-I mRNA levels was measured by RT-real time PCR at the indicated times after poly(I:C) transfection. The real time PCR values are an average of two independent reactions.

FIGS. 8a-8b show in vitro analysis of the interaction between the RNA helicase RIG-I and chemically synthesized siRNAs containing or not containing 2-nt 3′ overhangs. (FIG. 8a) In vitro binding and unwinding assay of a 27+0 or a 27+2 siRNA in the presence of increasing concentrations of full-length RIG-I. (FIG. 8b) Quantification of the dsRNA, ssRNA and the RNA/Protein complex formed in the presence of increasing amounts of RIG-I with the 27+0 or the 27+2 siRNA. The results are representative of two independent experiments.

### DETAILED DESCRIPTION OF THE INVENTION

As described herein, the response of mammalian cells to chemically synthesized siRNAs of different sizes containing different types of overhangs and end modifications was analyzed. While all of the siRNAs tested silenced the target gene (GFP), not all of them activated dsRNA-signaling pathways. While size of the siRNA molecule was a factor in inducing non-specific activation of dsRNA signaling, more importantly, it was found that the presence of the 2 nucleotide (2-nt) 3′ overhangs characteristic of Dicer products precluded activation of these pathways. These results demonstrate the structural basis for discrimination between products of the Dicer-mediated siRNA and miRNA pathways and products of viral infection.

In particular, the present invention is directed to a method of modulating activation of a double stranded RNA (dsRNA) signaling pathway, such as dsRNA that accompanies RNA interference (RNAi) of a (one or more) target RNA sequence, in a (one or more) cell or an individual, comprising introducing into the cell or individual small interfering RNA (siRNA) that degrades the target RNA sequence, and maintaining the cell or the individual under conditions in which RNAi of the target RNA sequence occurs and activation of the dsRNA signaling pathway is modulated in the cell or individual.

The methods described herein can be used in any suitable cell in which RNAi occurs such as mammalian cells, bacterial cells, viral cells, plant cells, fungal cells and parasitic cells. In addition the methods described herein can be used to produce the desired effects in individuals such as vertebrates (e.g., mammals such as primate (e.g., human), canine, feline, bovine, equine, rodent (e.g., mouse, rat)) and invertebrates.

As used herein the term “modulating” includes inducing (e.g., initiating activation), enhancing (e.g., enhancing an existing response), and inhibiting. In addition, the modulation of the activation of a dsRNA signaling pathway can be complete or partial (e.g., partial or complete inhibition of the dsRNA signaling pathway).

In one embodiment, the invention is directed to a method of inducing activation of a double stranded RNA (dsRNA) signaling pathway that accompanies RNA interference (RNAi) of a target RNA sequence in a cell, comprising introducing into the cell small interfering RNA (siRNA) that degrades the target RNA sequence, and maintaining the cell under conditions in which RNAi of the target RNA sequence occurs and activation of the dsRNA signaling pathway is induced in the cell. In this embodiment of the invention, the siRNA that is introduced into the cell can be double stranded and can comprise at least one blunt end. For example, the siRNA can be an siRNA wherein both ends are blunt-ended; an siRNA wherein one end is blunt-ended and the other end comprise a 5′ 2 nucleotide overhang; an siRNA wherein one end is blunt-ended and the other end comprises a 3′ 2 nucleotide overhang; and/or a combination thereof. Alternatively, in this embodiment, the siRNA that is introduced into the cell can be double stranded and comprise a 5′ 2 nucleotide overhang at each end. In a particular embodiment, an overhang can comprise from about 1 nucleotide to about 5 nucleotides.

The methods described herein can also be used to enhance the activation of an existing dsRNA signaling pathway in a cell (e.g., a cell infected with a virus). Thus, the present invention further provides a method of enhancing activation of a double stranded RNA (dsRNA) signaling pathway that accompanies RNA interference (RNAi) of a target RNA sequence in a cell, comprising introducing into the cell small interfering RNA (siRNA) that degrades the target RNA sequence, and maintaining the cell under conditions in which RNAi of the target RNA sequence occurs and activation of the dsRNA signaling pathway is enhanced in the cell. In this embodiment of the invention, the siRNA that is introduced into the cell can be double stranded and can comprise at least one blunt end. For example, the siRNA can be an siRNA wherein both ends are blunt-ended; an siRNA wherein one end is blunt-ended and the other end comprise a 5′ 2 nucleotide overhang; an siRNA wherein one end is blunt-ended and the other end comprises a 3′ 2 nucleotide overhang; and/or a combination thereof. Alternatively, in this embodiment, the siRNA that is introduced into the cell can be double stranded and comprise a 5′ 2 nucleotide overhang at each end. In a particular embodiment, an overhang can comprise from about 1 nucleotide to about 5 nucleotides.

In another embodiment, the invention is directed to a method of inhibiting (e.g., partially, completely) activation of a double stranded RNA (dsRNA) signaling pathway that accompanies RNA interference (RNAi) of a target RNA sequence in a cell, comprising introducing into the cell small interfering RNA (siRNA) that degrades the target RNA sequence, and maintaining the cell under conditions in which RNAi of the target RNA sequence occurs and activation of the dsRNA signaling pathway is induced in the cell. In this embodiment of the invention, the siRNA that is introduced into the cell can be double stranded and comprise at least 2 overhangs. An overhang can comprise from about 1 nucleotide to about 5 nucleotides. In a particular embodiment, each of the at least 2 overhangs comprise 2 nucleotides. In this embodiment of the invention, the siRNA can be an siRNA wherein both 3′ ends comprise a 2 nucleotide overhang; an siRNA wherein one end comprises a 3′ 2 nucleotide overhang and the other end comprises a 5′ 2 nucleotide overhang; and/or a combination thereof. Alternatively, or in addition to, the siRNA that can be used to inhibit activation of a dsRNA signaling pathway that accompanies RNAi of a target RNA sequence in a cell is an siRNA that comprises an oligodeoxynucleotide modification in one or more strands and/or at one or more ends of the siRNA. For example, one end of the siRNA can be blunt-ended and the other end can comprise an oligodeoxynucleotide (DNA) sequence on the other end (e.g., on one strand or both strands of a double stranded siRNA).

Various methods for determining or measuring activation of a dsRNA signaling pathway can be used in the methods of the present invention. Examples of such methods are provided herein and are known in the art. For example, measurement of p56 (p56 induction), PKR and/or RIG-I can be used to determine or measure activation of dsRNA signaling pathway in the methods of the present invention.

The invention is also directed to a method of degrading a target RNA sequence using RNA interference (RNAi) in the absence of a (one or more) non-specific effect (e.g., activation of a dsRNA signaling pathway) in a cell (degrading a target RNA sequence using RNAi wherein a dsRNA signaling pathway is not activated during RNAi). The method comprises introducing into the cell small interfering RNA (siRNA) that degrades the target RNA sequence, wherein the siRNA is double stranded and comprises at least 2 overhangs, and maintaining the cell under conditions in the which the target RNA sequence is degraded by the siRNA in the absence of non-specific effects.

Also encompassed by the present invention is a method of degrading a target RNA sequence using RNA interference (RNAi) in the presence of one or more non-specific effect in a cell (degrading a target RNA sequence using RNAi wherein a dsRNA signaling pathway is activated during RNAi). The method comprises introducing into the cell small interfering RNA (siRNA) that degrades the target RNA sequence, wherein the siRNA is double stranded and comprises at least one blunt end, and maintaining the cell under conditions in which the target RNA sequence is degraded by the siRNA in the presence of non-specific effects. In another embodiment, the siRNA is double-stranded and comprises a 5′ 2 nucleotide overhang at each end.

In one embodiment of the methods of degrading a target RNA sequence using RNAi in the absence or presence of a non-specific effect in a cell, the non-specific effect is activation of the double stranded RNA (dsRNA). In a particular embodiment, activation of the dsRNA signaling pathway results in an inflammatory response and/or apoptosis in the cell. Therefore, the methods of the present invention provide for degrading a target RNA sequence using RNAi in the absence or presence of an inflammatory response and/or apoptosis associated with activation of a dsRNA signaling pathway. For example, the methods described herein allow the skilled artisan to design an siRNA being used to treat an inflammatory condition (e.g., arthritis; use of RNAi with coated stents in patients with cardiovascular disease) such that upon administration of the siRNA, inflammation is inhibited (partially, completely). Alternatively, the methods described herein allow the skilled artisan to design an siRNA being used to treat a condition in which inflammation would be beneficial (e.g., viral infection, cancer) such that upon administration of the siRNA, inflammation is induced and/or enhanced (partially, completely).

Accordingly, the methods described herein can also be used to induce and/or enhance an antiviral or an anticancer effect in a cell or an individual using RNAi. The antiviral or anticancer effect in a cell or individual is induced and/or enhanced by using siRNA that degrades a (one or more) target RNA sequence and activates a dsRNA signaling pathway. The methods described herein can be used to produce an antiviral effect or an anticancer effect in an individual such as a mammal (e.g., primate, human), canine, feline, bovine, equine, rodent (e.g., mouse, rat).

In one embodiment, the present invention is also directed to a method of inducing and/or enhancing an antiviral effect using RNA interference (RNAi) in a cell, comprising introducing into the cell small interfering RNA (siRNA) that degrades a target RNA sequence of a virus (e.g., wherein the siRNA is double stranded and comprises at least one blunt end) and maintaining the cell under conditions in which the target RNA sequence of the virus is degraded by the siRNA in the presence of non-specific effects (e.g. promote dsRNA signaling pathway) in the cell. The present invention also provides for a method of inducing and/or enhancing an antiviral effect using RNA interference (RNAi) in an individual, comprising introducing into the individual (e.g., introducing (in vivo, ex vivo) into one or more cells of the individual) small interfering RNA (siRNA) that degrades a target RNA sequence of a virus, wherein the siRNA is double stranded and comprises at least one blunt end, under conditions in which the target RNA sequence of the virus is degraded by the siRNA in the presence of non-specific effects in the individual. The methods can be used to induce or enhance an antiviral effect against any virus being treated with siRNA (e.g., hepatitis virus, human immunodeficiency virus (HIV), and influenza virus).

In another embodiment, the invention also pertains to a method of inducing and/or enhancing an anticancer effect induced using RNA interference (RNAi) in a cell, comprising introducing into the cell small interfering RNA (siRNA) that degrades a target RNA sequence of a cancer (e.g., wherein the siRNA is double stranded and comprises at least one blunt end) and maintaining the cell under conditions in which the target RNA sequence of the cancer is degraded by the siRNA in the presence of non-specific effects (e.g., promote dsRNA signaling) in the cell. The methods described herein can be used to produce an anticancer effect in an individual. For example, the present invention provides for a method of inducing and/or enhancing an anticancer effect using RNA interference (RNAi) in an individual, comprising introducing into the individual (e.g., introducing (in vivo, ex vivo) into one or more cells of the individual) small interfering RNA (siRNA) that degrades a target RNA sequence of a cancer (e.g., wherein the siRNA is double stranded and comprises at least one blunt end and targets an RNA sequence produced by or associated with a tumor (e.g., an oncoprotein) in the individual) under conditions in which the target RNA sequence of the cancer is degraded by the siRNA in the presence of non-specific effects in the individual. The methods can be used to induce or enhance an anticancer effect against known cancers being treated with siRNA (e.g., breast cancer, prostate cancer, ovarian cancer, melanoma, leukemia, Hodgkin's disease).

According to the methods of the present invention, the method can be used therapeutically (e.g., in order to treat an individual that has been infected with the virus or has developed the cancer that is being targeted), or prophylactically (e.g., in order to protect an individual against becoming infected with the virus or developing the cancer that is being targeted).

The siRNA for use in the methods of the present invention can be synthesized, for example, using the methods described herein or obtained from commercial sources. In one embodiment, the siRNA is double stranded and can comprise a sequence that is from about 19 nucleotides to about 30 nucleotides. In particular embodiments, the siRNA is double stranded and can comprise a sequence that is from about 21 nucleotides to about 27 nucleotides; or from about 23 to about 25 nucleotides. In other embodiments, the siRNA is double stranded and one or both strands (e.g., sense, antisense) can comprises a sequence of about 19, 20, 21, 22, 23, 24, 25, 26, 27, 28, 29, 30, 31, 32, 33, 34 or 35 nucleotides. In a particular embodiment, the siRNA is double stranded and both strands comprise a sequence that is about 27 nucleotides. The siRNA is comprised of RNA, and in some embodiments, can include DNA base pairs, either at the end of or within one or more of the strands of the siRNA.

As described herein the siRNA can comprise one or more blunt ends and/or one or more overhangs. Preparation of siRNA that comprise one or more blunt ends and/or one or more overhangs can be prepared using methods described herein or known in the art. The overhangs can be present on one or more strands of a double stranded siRNA. In one embodiment, an overhang can comprise from about 1 to about 5 nucleotides. In another embodiment, the overhand comprises 1, 2, 3, 4 or 5 nucleotides. In a particular embodiment, the overhang comprises 2 nucleotides. The overhang can comprise RNA, and in some embodiments, DNA base pairs.

In the embodiments in which activation of a double stranded RNA (dsRNA) signaling pathway that accompanies RNA interference (RNAi) of a target RNA sequence is induced, the siRNA can be double stranded and can comprise at least one blunt end. For example, the siRNA can be an siRNA wherein both ends are blunt-ended; an siRNA wherein one end is blunt-ended and the other end comprise a 5′ 2 nucleotide overhang; an siRNA wherein one end is blunt-ended and the other end comprises a 3′ 2 nucleotide overhang; and/or a combination thereof. Alternatively, in this embodiment, the siRNA that is introduced into the cell can be double stranded and comprise a 5′ 2 nucleotide overhang at each end. In a particular embodiment, an overhang can comprise from about 1 nucleotide to about 5 nucleotides.

In the embodiment in which activation of a double stranded RNA (dsRNA) signaling pathway that accompanies RNA interference (RNAi) of a target RNA sequence is inhibited, the siRNA can be double stranded and comprise at least 2 overhangs. An overhang can comprise from about 1 nucleotide to about 5 nucleotides. In a particular embodiment, each of the at least 2 overhangs comprise 2 nucleotides. In this embodiment of the invention, the siRNA can be an siRNA wherein both 3′ ends comprise a 2 nucleotide overhang; an siRNA wherein one end comprises a 3′ 2 nucleotide overhang and the other end comprises a 5′ 2 nucleotide overhang; and/or a combination thereof. Alternatively, or in addition to, the siRNA comprises an oligodeoxynucleotide modification in one or more strands and/or at one or more ends of the siRNA. For example, one end of the siRNA can be blunt-ended and the other end can comprise an oligodeoxynucleotide (DNA) sequence on the other end (e.g., on one strand or both strands of a double stranded siRNA).

As used herein, the “target RNA sequence” is any RNA sequence in a cell or in an individual that is selected to be degraded using RNAi. Many such sequences are known in the art. For example, the RNA sequence can be a viral sequence or a sequence of a protein that is associated with a cancer, such as an oncoprotein.

### Exemplification

### Methods

Cells and reagents.

T98G, HT1080, Hela, and 293T cells were grown in DMEM media supplemented with 10% fetal bovine serum (FBS) at 37° C. in a 5% CO₂ atmosphere. Stable pools of T98G, HT1080 and Hela cells expressing enhanced green fluorescent protein (GFP) were generated by transduction with lentiviral vectors containing the GFP gene under the control of a CMV promoter as previously described (Marques, J. T. et al., J Virol. 79, 11105-14 (2005)). Antibody against GFP was from Roche Diagnostics, α-Tubulin, Flag-M2 and β-Actin were from Sigma, and GAPDH was from Chemicon International. Antibodies against p56 were a gift from Ganes Sen. Poly riboinosinic-ribocytidylic acid (I:C) was purchased from Sigma. The TAR₅₇ RNA was synthesized by in vitro transcription using T7 RNA polymerase as described (Carpick, B. W. et al., J Biol Chem. 272, 9510-6 (1997)).

siRNAs.

All the siRNAs used in this study were chemically synthesized by Dharmacon or Integrated DNA Technologies (IDT) as indicated in Table 1 based on previously described sequences (Kim, D. H. et al., Nat Biotechnol. 23, 222-6 (2005); Rose, S. D. et al., Nucleic Acids Res. 33, 4140-56 (2005)). The single strands were annealed by pre-heating at 90° C. for 1 min followed by a 1 h incubation at room temperature. For verification, all the single strands were separated in a denaturing 15% polyacrylamide gel containing 8M Urea and the annealed duplexes were separated in a native 10% polyacrylamide gel. The RNA was visualized by staining the gel with SYBR Gold (Molecular Probes).

### Transfections.

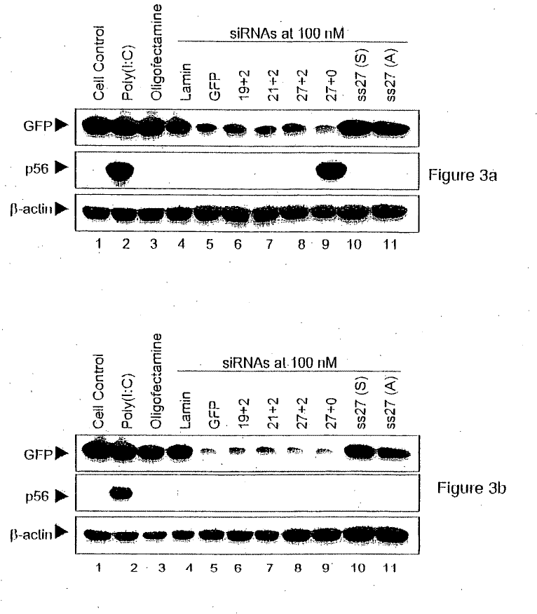

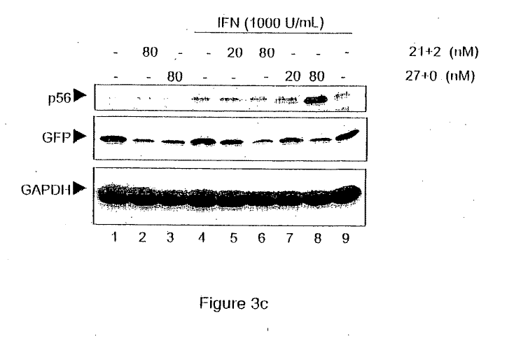

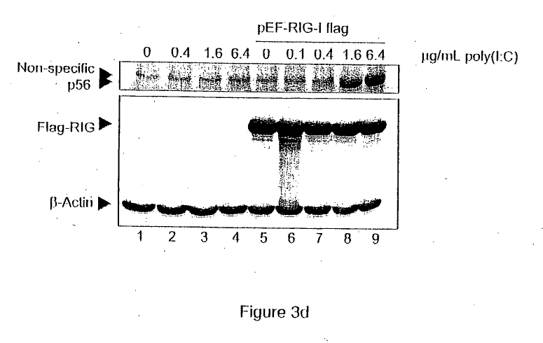

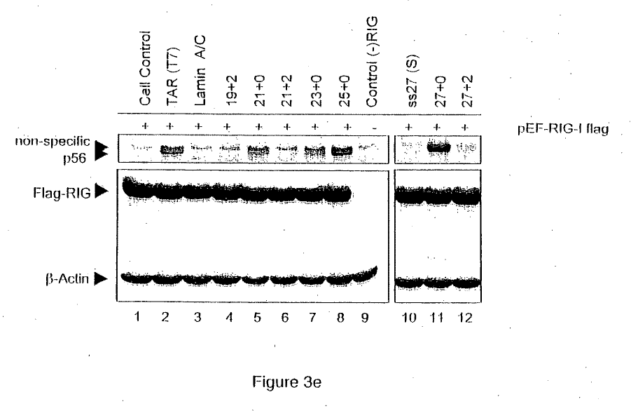

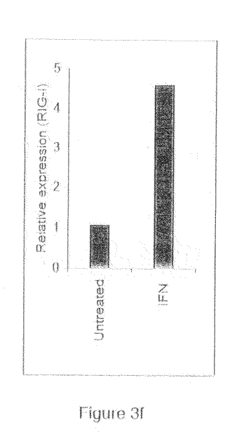

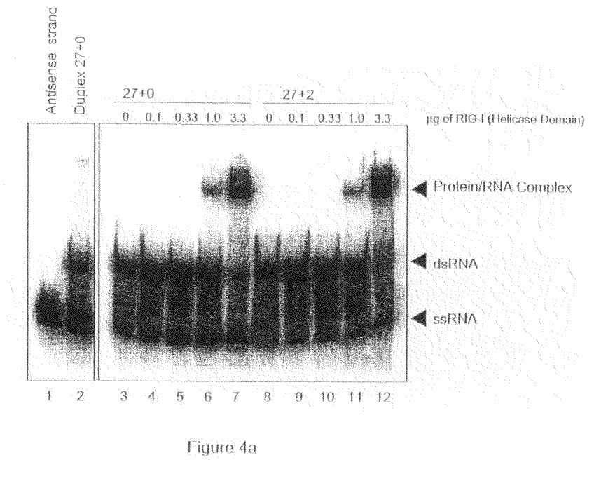

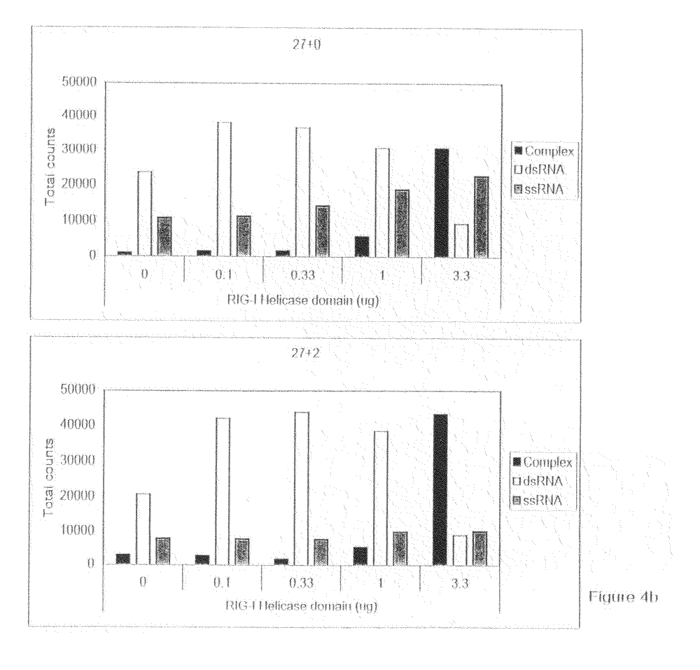

siRNAs were transfected using Oligofectamine (Invitrogen) in T98G (only FIG. 3a), HT1080 and Hela cells, or Dharmafect #2 (Dharmacon) in T98G (all the other figures) and 293T cells according to the manufacturer's protocols. In all cases the reagent was first dissolved in OptiMEM for 5 min before being mixed with the same volume of OptiMEM containing the siRNA. The ratio used was 2.5 μL of reagent per 1.5 μg of siRNA unless indicated. Plasmid transfections in 293T cells were performed using Lipofectamine (Invitrogen) according to the manufacturer's protocols.

### Western Blot.

The western blots were performed as described (Marques, J. T. et al., J Virol. 79, 11105-14 (2005)). Cells were lysed in 50 mM Tris buffer, pH 7.4 containing 150 mM of NaCl, 50 mM of NaF, 10 mM of β-glycerophosphate, 1% Triton X-100, 0.1 mM of EDTA, 10% glycerol and protease/phosphatase inhibitors and protein concentrations were determined using the Protein assay kit (Bio-Rad).

### Fluorescence Assays.

Fluorescence in the lysates was determined in duplicates using a Victor³V (Perkin-Elmer). The fluorescence values were averaged, normalized to total protein concentration and plotted as a percentage of the fluorescence read from the control.

### RNA Extractions.

For the Northern Blot, total RNA was extracted form 10 cm dishes using the TRIZOL reagent according to the protocols provided by the manufacturer (Invitrogen).

### Reverse Transcription Polymerase Chain Reaction.

Total RNA was reverse transcribed into cDNA using poly(A) priming and Superscript II (Life Technologies, Inc.) reverse transcriptase according to the manufacturer's instructions. Real-time PCR analysis was performed using SYBR-Green (PerkinElmer) according to the manufacturer's instructions with the specific primer pairs for

| | |
|---|---|
| human ISG56 | |
| 5′ GCCACAAAAAATCACAAGCCA 3′ | (SEQ ID NO: 1) |
| and | |
| 5′ CCATTGTCTGGATTTAAGCGG 3′, | (SEQ ID NO: 2) |
| human RIG-I | |
| 5′GACTGGACGTGGCAAAACAA 3′ | (SEQ ID NO: 3) |
| and | |
| 5′ TTGAATGCATCCAATATACACTTCTG 3′ | (SEQ ID NO: 4) |
| and | |
| human GAPDH | |
| 5′ GAAGGTGAAGGTCGGAGT 3′ | (SEQ ID NO: 5) |
| and | |
| 5′ GAAGATGGTGATGGGATTTC 3′. | (SEQ ID NO: 6) |

The two-temperature cycle of 95° C. for 15 s and 60° C. for 1 min (repeated for 40-45 cycles) was used, and Ct was measured using the iCycler (Bio-Rad). Relative transcript quantities were calculated by the ΔΔCt method using GAPDH as a reference. Normalized samples were then expressed relative to the average ΔCt value for untreated controls to obtain relative fold change in expression levels.

### Helicase and Binding Assay.

The antisense strand of the siRNA was labeled using T4 polynucleotide kinase (Invitrogen) and γ-³²P ATP (Amersham Biosciences) according to the manufacturer's protocol. The labeled antisense strand was annealed to unlabeled sense strand as described above. 3 ng of the labeled siRNA duplex was incubated with various concentration of RIG-I, full length of the helicase domain alone, (0.1˜3.3 μg) in the helicase buffer (25 mM Tris, pH 7.4, 3 mM MgCl₂, 3 mM DTT, 2 mM ATP) for 1 h at room temperature in a 20 μL volume. 2 μl of 50% Glycerol was added and the samples were centrifuged at 10,000 g for 1 min before being separated in a native 10% polyacrylamide gel in TBE buffer. The gel was dried at 80° C. for 1 h (Bio-Rad Gel Dryer) and exposed on a phospho-imager screen (Molecular Dynamics) that was scanned using a Storm 840 (General Electric) and analyzed using the ImageQuant 1.2 software (Molecular Dynamics).

### ATPase Assay.

ATPase assays were performed in the helicase buffer (25 mM Tris, pH 7.4, 3 mM MgCl₂, 3 mM DTT, 2 mM ATP) with 4 μg of RIG-I full length and 1 μg of siRNA in a 20 μL reaction for 1 h at 37° C. Malachite green solution (Bio-Rad) was added for 5 min and the absorbance at 670 nm was determined.

### Immunoaffinity-Purification of RIG-I.

293T cells overexpressing Flag-RIG-I were lysed in 50 mM Tris, pH 7.4, 1 mM EDTA, 1% NP-40, 150 mM NaCl buffer. The lysate was incubated overnight at 4° C. with Ezview™ Red ANTI-FLAG M2 Affinity Gel (Sigma) according to the protocols provided by the manufacturer. The gel was washed extensively before the elution of the Flag-fusion protein using an excess of Flag peptide in 25 mM Tris, pH 7.4, 3 mM MgCl₂ buffer.

### Cytotoxicity Assay.

T98G cells were transfected in triplicates with the siRNAs in 96-well plates. After 5 days, cells were fixed with 10% formaldehyde in Phosphate buffered saline and stained with 1% Crystal Violet. The absorbance in each well was determined using a Victor³V (Perkin-Elmer).

### Results

Differential Activation of RNAi and dsRNA Signaling Pathways by siRNAs

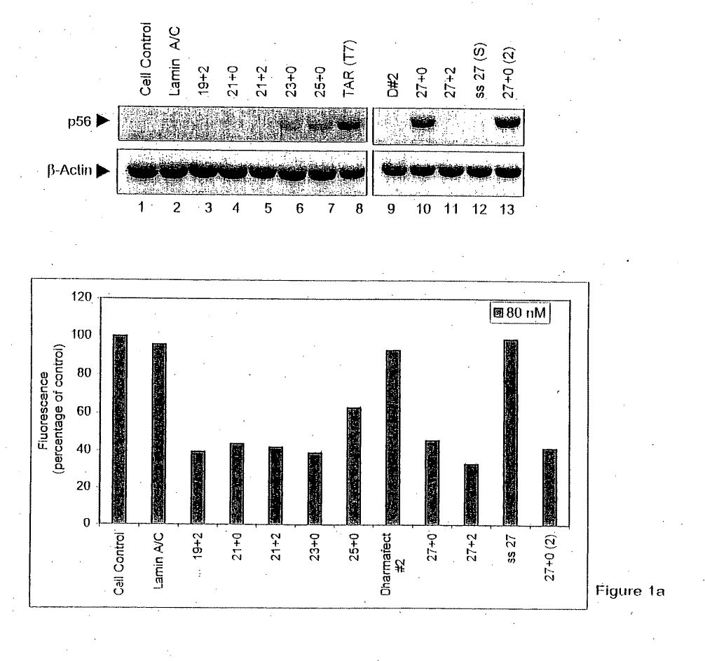

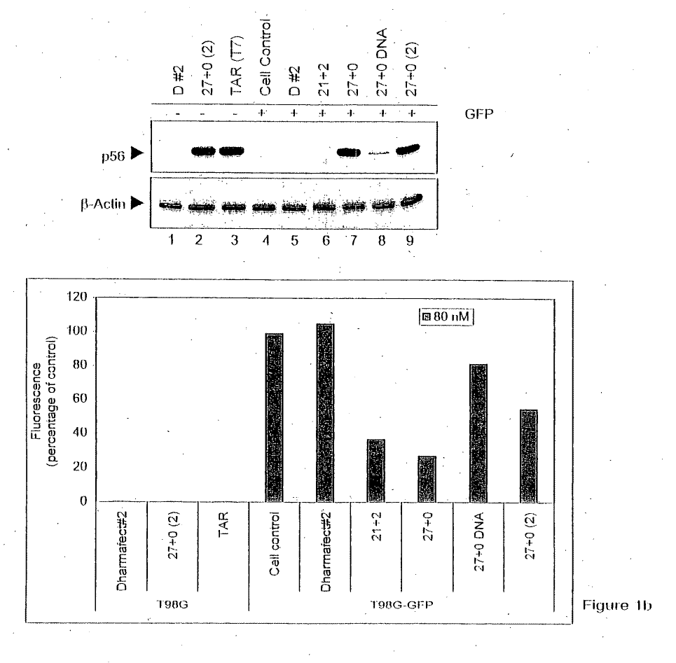

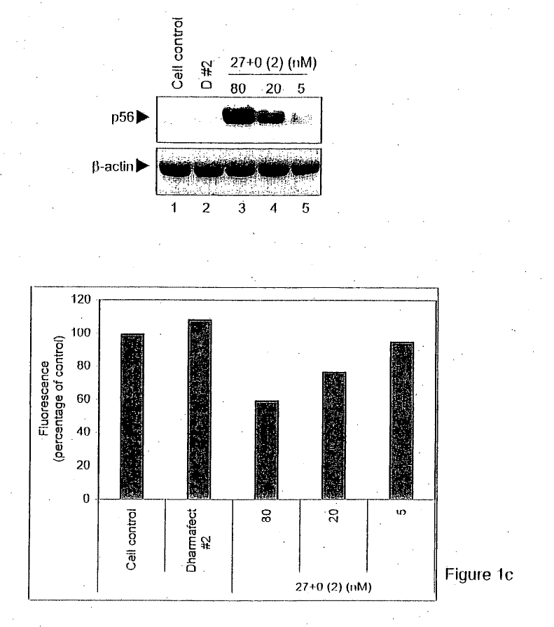

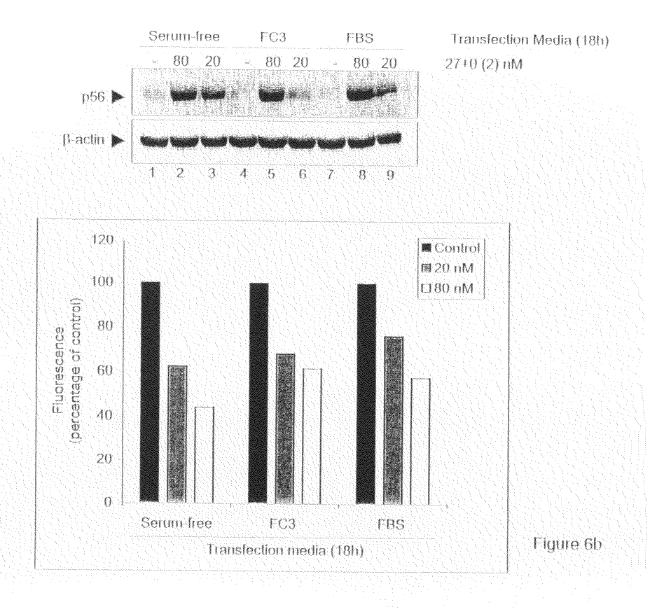

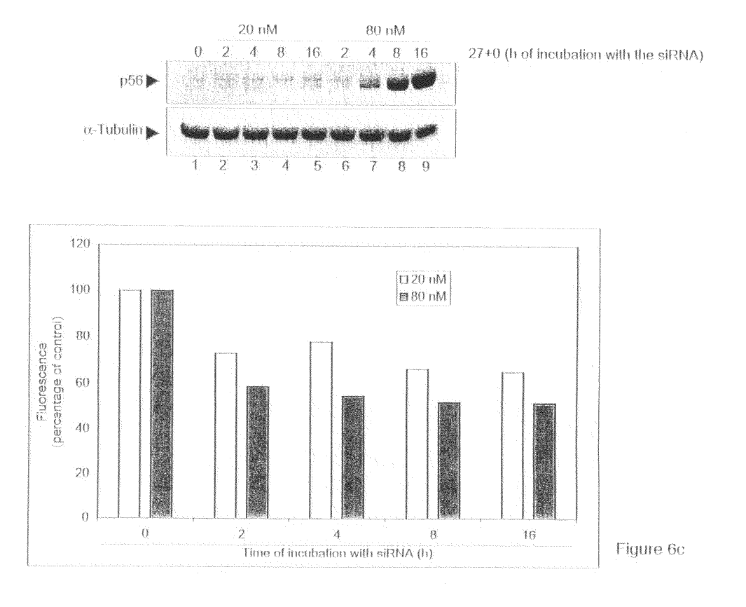

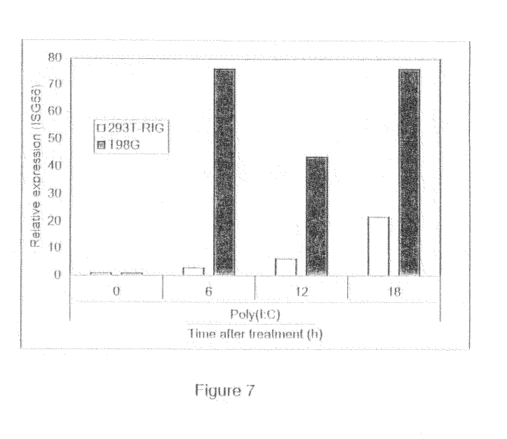

In order to understand the activation of mammalian dsRNA signaling pathways by siRNA, RNA duplexes of different lengths containing blunt ends or 3′ overhangs previously shown to be effective at triggering RNAi (Table 1) (Kim, D. H. et al., Nat Biotechnol. 23, 222-6 (2005)) were tested in the glioblastoma cell line, T98G. T98G cells are very sensitive to the non-specific activation of the IFN system by siRNA (Sledz, C. A., et al., Nat Cell Biol. 5, 834-9 (2003)). Stable pools of T98G cells expressing GFP were generated in order to determine specific silencing of the GFP target gene as well as any non-specific effects. One measure of activation of dsRNA signaling pathways is expression of the dsRNA-induced protein 56K (p56). p56 is a sensitive indicator of the activation of IRF-3, which is independent of IFN production (Sledz, C. A., Holko, M., de Veer, M. J., Silverman, R. H., Williams B. R., Nat Cell Biol. 5, 834-9 (2003); Peters, K. L., et al., Proc Natl Acad Sci USA. 99, 6322-7 (2002)). Although all of the siRNAs tested were capable of silencing GFP expression, their capacity to induce p56 varied greatly (FIG. 1a). The level of induction of p56 increased with the length of the siRNA. Thus, only minimal induction was observed with a 23 nt (23+0) siRNA, but increasing levels of induction were seen with 25 (25+0) and 27 (27+0) nt siRNAs (FIG. 1a). Since the presence of a 5′-triphosphate on RNAs synthesized using phage polymerases is a potent inducer of IFN production (Kim, D. H. et al., Nat Biotechnol. 22, 321-5 (2004)), a T7 RNA polymerase synthesized RNA (TAR) was used as a positive control for the induction of dsRNA signaling. The induction of p56 by the 27+0 siRNA was comparable to the induction observed with TAR, indicating the potency of the siRNA molecules at triggering dsRNA signaling (FIG. 1a, compare lanes 8 and 10). Interestingly, at the same concentration of 27+0 siRNA that induced high levels of p56 (80 nM), a 27+2 version of the same siRNA containing a 2-nt 3′ overhang did not induce any detectable protein (FIG. 1a, compare lanes 10 and 11). Moreover, an siRNA with the same length and structure (referred to as 27+0(2)) but targeting a different region of the GFP sequence induced the same levels of p56 as the original 27+0 (FIG. 1a, compare lanes 10 and 13). Thus, the induction of p56 appears to be related to the end structure of the siRNA rather than its sequence or length. Therefore, this response is different from the Toll like receptor (TLR)-dependent sequence-specific induction of IFN recently described in immune cells treated with siRNAs (Hornung, V. et al., Nat Med. 11, 263-70 (2005); Judge, A. D. et al., Nat Biotechnol. [Epub ahead of print] (2005)). Indeed, unlike the production of IFN by immune cells (Hornung, V. et al., Nat Med. 11, 263-70 (2005)), the induction of p56 by siRNAs could not be blocked by chloroquine, an inhibitor of endosomal maturation (FIG. 6a). A DNA version of the 27+0 molecule was a much weaker inducer of p56 than the RNA equivalent (FIG. 1b, compare lanes 7 and 8). Moreover, the induction of p56 did not require the expression of the mRNA target of the siRNA since wild-type (non-GFP expressing) T98G cells induced the same levels of p56 in response to the 27+0(2) siRNA as GFP expressing T98G cells (FIG. 1b, compare lanes 2 and 9). Both silencing of GFP and induction of p56 by the siRNAs was concentration dependent (FIG. 1c). At a 5 nM concentration, there was no significant induction of p56 and no GFP silencing by the 27+0(2) siRNA. Although it should be noted that using more potent siRNA resulted in silencing at concentrations where p56 induction was not detected (data not shown). However, with the 27+0 siRNA, a consistent correlation between the extent of RNAi-mediated gene silencing and of activation of dsRNA signaling was observed. In all cases where increased silencing of GFP was observed, such as using serum-free media for the transfection or by increasing the transfection time, there was a corresponding increase in p56 induction (FIG. 6b, FIG. 6c). These results suggest that increased delivery of the siRNA to the cytoplasm enhances both RNAi and dsRNA signaling.

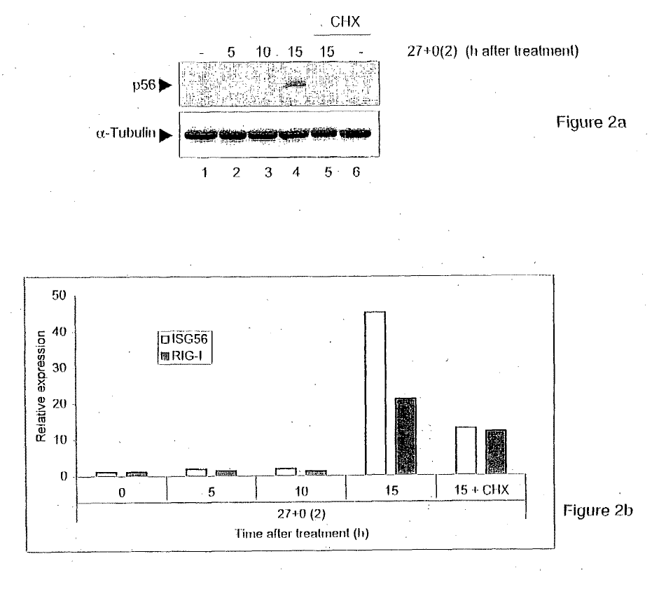

The induction of p56 by long dsRNA molecules is a primary event independent of protein synthesis that occurs through activation of IRF-3 independently of IFN action (Peters, K. L., et al., Proc Natl Acad Sci USA. 99, 6322-7 (2002)). To determine whether IFN plays a role in the induction of p56 by bluntended siRNAs, T98G cells were treated with the 27+0 siRNA in the presence or absence of cycloheximide (CHX) to inhibit protein synthesis. Total RNA and protein were prepared at different times after treatment and analyzed for the induction of ISG56 mRNA (which encodes p56) and p56 protein, respectively. Although induction of both ISG56 mRNA and p56 protein was detectable only after prolonged (15 h) treatment with siRNA (FIG. 2a, FIG. 2b), characteristic of a secondary response, the fact that CHX treatment did not block induction of the ISG56 mRNA suggests otherwise. CHX treatment did block de novo protein synthesis as monitored by the induction of p56 (FIG. 2a, compare lanes 4 and 5). The reduction seen in ISG56 mRNA induction following CHX treatment is likely a result of the absence of an IFN feedback mechanism that amplifies gene induction following exposure to dsRNA (Marie, I., et al., EMBO J. 17, 6660-9 (1998); Hata, N. et al., Biochem Biophys Res Commun. 285, 518-25 (2001); Nakaya, T. et al., Biochem Biophys Res Commun. 283, 1150-6 (2001); Elco, C. P., et al., J Virol. 79, 3920-9 (2005)). Nevertheless, it is clear that the primary induction of p56 by siRNA is IFN-independent and does not require protein synthesis.

The RNA Helicase RIG-I is Involved in the Activation of dsRNA Signaling Pathways by siRNAs

Cell lines commonly used in laboratories have varying abilities to respond to different sources of dsRNA. For example, in contrast to T98G cells, p56 can be induced in Hela cells in response to poly(I:C) and T7 synthesized RNAs (Kim, D. H. et al., Nat Biotechnol. 22, 321-5 (2004)) but not in response to the 27+0 siRNA (FIGS. 3a-3b, compare FIG. 3a and FIG. 3b and unpublished observations). Furthermore, even T98G cells show a marked difference in the time course of induction of p56 in response to the 27+0 siRNA and poly(I:C), with the latter showing much faster kinetics with maximum mRNA levels already detected at 6 h post-treatment (compare FIG. 2b and FIG. 7). HT1080 cells mount a robust response to poly(I:C) (Peters, K. L., et al., Proc Natl Acad Sci USA. 99, 6322-7 (2002)) and T7 synthesized RNAs (data not shown) but did not show any p56 induction in response to the 27+0 siRNA (FIG. 3c). However, pre-treatment of HT1080 cells with IFN could restore their ability to produce p56 in response to the 27+0 siRNA (FIG. 3c, compare lanes 3 and 8). Importantly, even after IFN pre-treatment, only the 27+0 siRNA but not a traditional 21+2 siRNA could induce p56 (FIG. 3c, compare lanes 6 and 8). The response of the cells to the IFN treatment can be observed by the induction of p56 itself in all treated samples despite the higher levels in the cells transfected with the 27+0 siRNA (FIG. 3c). These results suggest a different mechanism than previously observed for the induction of IFN by poly(I:C) and by T7 synthesized RNAs (Sledz, C.A., Holko, M., de Veer, M. J., Silverman, R. H., Williams B. R., Nat Cell Biol. 5, 834-9 (2003); Kim, D. H. et al., Nat Biotechnol. 22, 321-5 (2004)).

In 293T cells, p56 induction was not observed, even in response to poly(I:C) (FIG. 3d). The response to poly(I:C), however, could be restored by overexpression of the IFN-inducible RNA helicase RIG-I, recently described as a dsRNA sensor capable of triggering IFN production (Yoneyama, M. et al., Nat Immunol. 5, 730-7 (2004)). Importantly, overexpression of RIG-I in 293T cells also restored their response to chemically synthesized siRNAs. Moreover, even a 21+0 siRNA was capable of inducing p56 in these cells but the presence of 2-nt 3′ overhangs again precluded the activation of dsRNA signaling similarly to T98G cells (FIG. 3e). In addition, the mRNA for RIG-I is also induced in HT1080 cells treated with IFN and in T98G cells transfected with the 27+0 siRNA (FIGS. 2b and 3f). Taken together, these data indicate that RIG-I is involved in dsRNA signaling by blunt ended siRNAs.

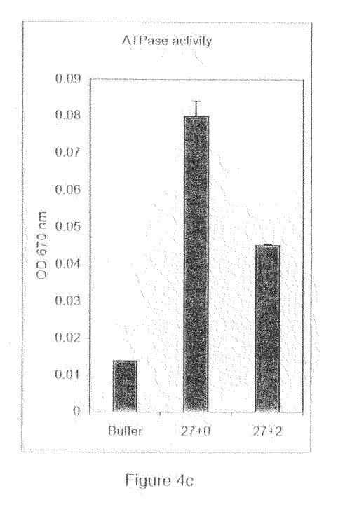

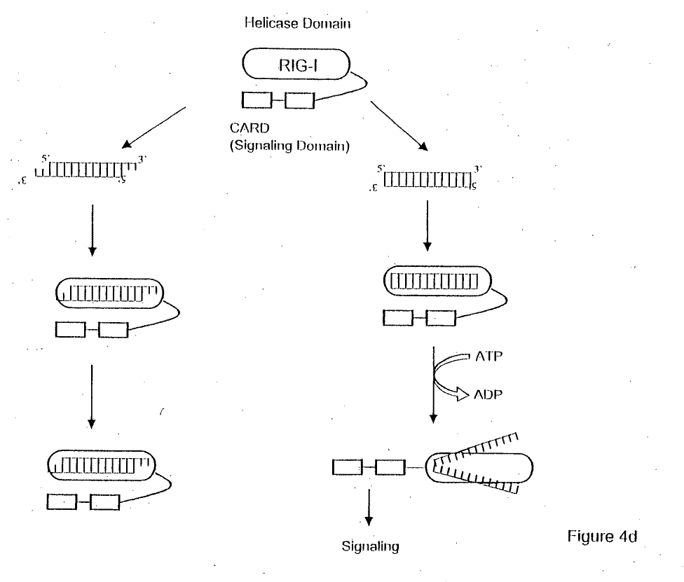

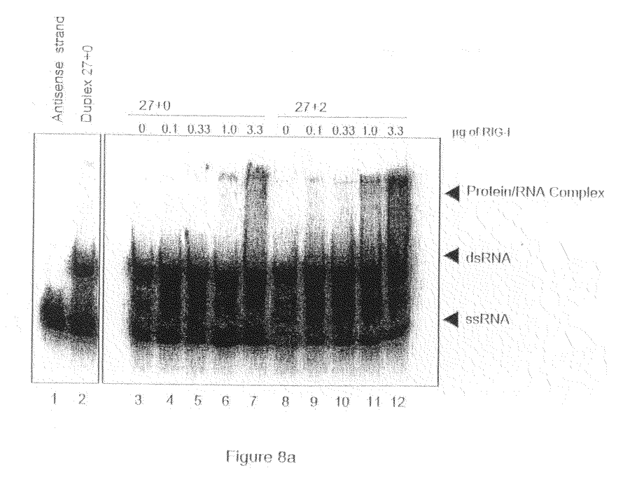

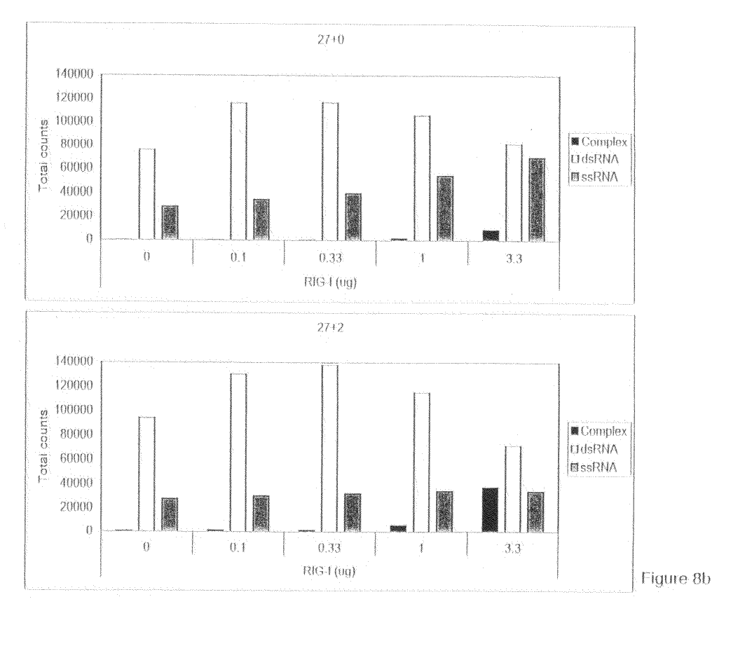

To investigate the mechanism by which RIG-I can differentiate between siRNAs with or without 3′ overhangs, in vitro binding and unwinding assays were performed to analyze the helicase activity of immunoaffinity-purified RIG-I on radioactively labeled substrate siRNAs. Although both the 27+0 and the 27+2 siRNAs bound the helicase domain of RIG-I in vitro with similar affinities, the 27+0 siRNA was more efficiently unwound resulting in increased release of the single strands from the RNA duplex (FIG. 4a, compare lanes 3-7 to lanes 8-12). Quantification of the relative amounts of the RNA/protein complex, the dsRNA and the ssRNA illustrates the differences in the interaction between RIG-I and the 27+0 or the 27+2 siRNAs (FIG. 4b). The same experiments were performed with full length RIG-I with essentially the same results apart from the RNA/protein complex being less clearly observed in the native gels (FIG. 8a, FIG. 8b). This is likely due to the presence of the CARD domains that can mediate dimerization or polymerization of RIG-I molecules generating high molecular weight complexes that do not enter the gel.

RNA helicases catalyze the cleavage of ATP to promote multiple rounds of unwinding (Tanner, N. K., Linder, P., Mol Cell. 8, 251-62 (2001)). Therefore, it was determined the ATPase activity of RIG-I alone or in the presence of the 27+0 or the 27+2 siRNAs. Consistent with the notion that the 27+0 is more efficiently unwound than the 27+2 siRNA, significantly higher ATPase activity was detected when RIG-I was incubated with the 27+0 siRNA than when it was incubated with the 27+2 siRNA (FIG. 4c).

Modulating the Activation of RNAi and dsRNA Signaling Pathways with Structure and Chemical Modifications of siRNAs

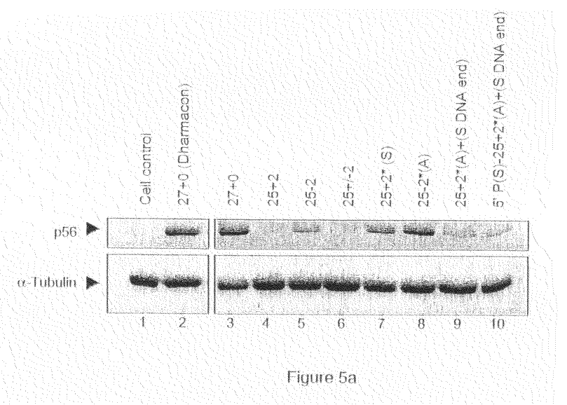

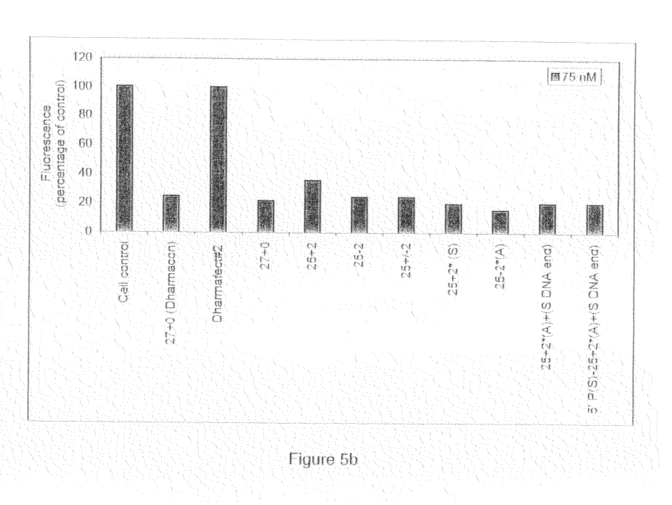

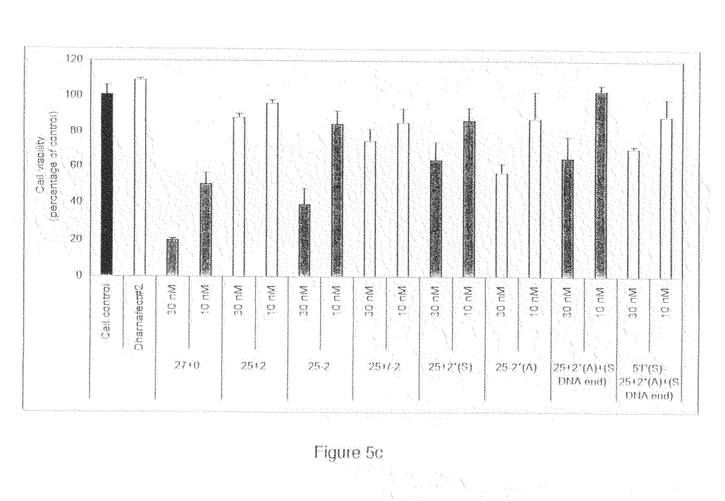

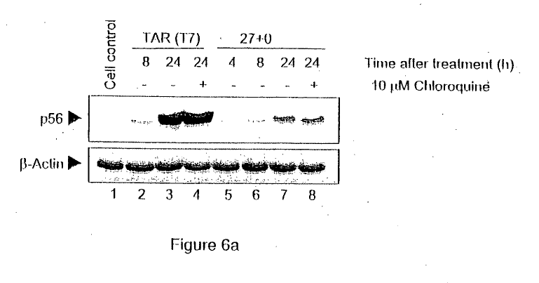

The data indicated that the extremities of the siRNA duplex are critical determinants of the capacity of a siRNA molecule to trigger dsRNA-signaling pathways. Regardless of the presence of 2-nt 3′ overhangs, all the duplexes tested were capable of triggering RNAi as observed by others (Kim, D. H. et al., Nat Biotechnol. 23, 222-6 (2005); Czauderna, F. et al., Nucleic Acids Res. 31, 2705-16 (2003)). These longer (27 nt) siRNAs are better substrates for Dicer and likely this increases the efficiency by which these siRNAs are incorporated into the RISC (Kim, D. H. et al., Nat Biotechnol. 23, 222-6 (2005); Siolas, D. et al., Nat Biotechnol. 23, 227-31 (2005)). However, it would not be desirable if this improvement were accompanied by increased activation of dsRNA signaling resulting in non-specific effects. Recently, new design approaches were described that improved the performance of longer siRNAs as Dicer substrates (Rose, S. D. et al., Nucleic Acids Res. 33, 4140-56 (2005)). The new designs included asymmetric siRNAs with a blunt end containing deoxyribonucleotides at selected positions and a 2-nt 3′ overhang on the other end. These modifications directed the way Dicer processed the dsRNA substrate, resulting in a more homogeneous pool of siRNAs products (Rose, S. D. et al., Nucleic Acids Res. 33, 4140-56 (2005)). Therefore, the effects of these modifications on the activation of dsRNA signaling by siRNAs (Table 1, Synthesized by IDT) were assessed. In T98G cells, the 27+0 siRNA was the strongest inducer of p56 followed closely by an asymmetric siRNA with one blunt end and a 2-nt 5′ overhang at the other end (25-2*(A)) (FIG. 5a, lanes 3 and 8). An asymmetric siRNA with one blunt end and a 2-nt 3′ overhang at the other end (25+2* (S)) and one with 2-nt 5′ overhangs at both ends (25-2) also induced significant amounts of p56 (FIG. 5a, lanes 5 and 7). Interestingly, asymmetric siRNAs with 3′ deoxynucleotides modifications on the blunt end and a 2-nt 3′ overhang on the other end (25+2*(A)+(S DNA end)) showed residual p56 induction when compared to the same design without the deoxynucleotides (25+2* (S)) (FIG. 5a, compare lanes 9 and 10 to lane 7). This is significant as this design of duplex was concluded to be the optimal substrate for Dicer from the standpoint of processing and functional potency and was recommended for general use (Siolas, D. et al., Nat Biotechnol. 23, 227-31 (2005)). Together with the observation that a DNA duplex is not an effective trigger for dsRNA signaling (FIG. 1b), these data confirm that RIG-I dependent signaling is selective for RNA (Yoneyama, M. et al., Nat Immunol. 5, 730-7 (2004)). The only siRNAs that did not induce detectable levels of p56 were those with 2-nt 3′ overhangs at both ends (25+2) or a 2-nt 3′ overhang at one end and a 2-nt 5′ overhang on the other (25+/−2) (FIG. 5a, lane 4 and 6). Again, despite the differential induction of p56, all of the siRNAs were capable of silencing GFP expression (FIG. 5a, FIG. 5b). These results establish that a blunt end is the strongest terminal structure for promoting activation of dsRNA signaling, followed by a 5′ overhang. In contrast, 3′ overhangs allow RNAi to proceed without activation of dsRNA signaling.

In mammalian cells, dsRNA signaling results in the induction of apoptosis (Marques, J. T. et al., J Virol. 79, 11105-14 (2005)). It was observed that the induction of p56 by the siRNAs described above correlated with the toxicity associated with each of the duplexes (FIG. 5, compare FIG. 5a and FIG. 5c). The most toxic siRNA was the 27+0 siRNA while the least toxic was the 25+2 siRNA (FIG. 5c). Given the link between dsRNA signaling and apoptosis, these varying degrees of toxicity support our conclusions regarding the induction of dsRNA signaling by the various siRNAs.

### Discussion

The discovery of mammalian miRNAs indicates that dsRNA is not exclusively produced by viruses. Therefore, mammalian cells must have mechanisms that allow them to discriminate between self from non-self RNAs. Different modes of discrimination are likely to exist. Indeed, the presence of a 5′ triphosphate is a critical feature of T7 synthesized RNAs that determines the activation of dsRNA signaling (Kim, D. H. et al., Nat Biotechnol. 22, 321-5 (2004)). Since siRNAs and miRNAs have a 5′ monophosphate they are inefficient in this regard (Elbashir, S. M., et al., Genes Dev. 15, 188-200 (2001); Cullen, B. R., Mol Cell. 16, 861-5 (2004)). The data indicates that both the size of the siRNA and the structure of the ends of the RNA duplex play important roles in the activation of dsRNA signaling. Although it was initially postulated that a minimum of 30 bp were necessary to activate dsRNA-signaling (Elbashir, S. M. et al., Nature. 411, 494-8 (2001); Caplen, N. J., et al., Proc Natl Acad Sci USA. 98, 97427 (2001)), it was found that as few as 21-23 bp are sufficient (FIG. 1a and FIG. 3f). The 30 bp limit was suggested from observations of the minimum size required to activate PKR in vitro (Manche, L., et al., Mol Cell Biol. 12, 5238-48 (1992)). However, it is shown here that siRNAs can activate dsRNA signaling through the RNA helicase RIG-I, likely independently of PKR. The data shows that the structures of the siRNA ends are the key determinants of this mode of activation. Pre-miRNAs generated by Drosha, as well as miRNAs and siRNAs generated by Dicer, have a characteristic 2-nt 3′ overhang (Elbashir, S. M., et al., Genes Dev. 15, 188-200 (2001); Cullen, B. R., Mol Cell. 16, 861-5 (2004)). It is shown that the presence of the 3′ overhangs precludes activation of dsRNA signaling by siRNAs and therefore provides an important basis for the discrimination between self and non-self dsRNAs. Interestingly, this type of discrimination appears to take place after binding of the dsRNAs to RIG-I. While overhangs do not affect RNA binding to the helicase, they do reduce the efficiency of its ATPase activity required for unwinding of the RNA duplex (FIG. 4). The current model indicates that after unwinding its substrate, RIG-I exposes its CARD homology domains, which leads to downstream signaling (FIG. 4d) (Yoneyama, M. et al., Nat Immunol. 5, 730-7 (2004); Levy, D. E., et al., Nat Immunol. 5, 699-701 (2004)). Consequently, it would be expected that a better substrate for the helicase activity of RIG-I would be a more efficient activator of dsRNA signaling. This expectation has been confirmed by our results comparing the 27+0 and 27+2 siRNAs.

siRNAs are emerging as potentially important therapeutic tools in the treatment of cancer, virus infections and other diseases (Zhang, W. et al., Nat Med. 11, 56-62 (2005)). In some clinical situations activation of dsRNA signaling and attendant apoptosis as a side effect of RNAi might be desirable, helping to eliminate cancer cells or virus-infected cells (Marques, J. T. et al., J Virol. 79, 11105-14 (2005)). In other situations, however, these non-specific effects and accompanied by proinflammatory responses would be detrimental to the desired therapeutic effect. Importantly, the results suggest that the activation of dsRNA signaling that accompanies RNAi can be modulated through chemical modifications of the RNA duplex used (FIG. 5a, FIG. 5c).

It is becoming clear that RNAi does serve as an antiviral mechanism in mammals, but it remains uncertain whether the RNAi and classical dsRNA signaling pathways engage in crosstalk (Chen, W., et al., FEBS Lett. 579, 2267-72 (2005); Bennasser, Y, et al., Immunity. 22, 607-19 (2005)). RIG-I is highly similar to Dicer Related Helicase 1 (DRH1) which is involved in processing long dsRNAs into siRNAs in C. elegans and thus has an important role in the recognition of long dsRNAs and possibly in antiviral defense in this organism (Tabara, H., et al., Cell. 109, 861-71 (2002)). RIG-I is a key dsRNA sensor involved in the antiviral response in mammals (Kato, H. et al., Immunity. 23, 19-28 (2005)). Thus, this family of helicases is clearly positioned at the top of antiviral pathways. Interestingly, other C. elegans proteins involved in RNAi, RDE-4 and RDE-3, are similar to the mammalian protein activator of PKR (PACT) and oligoadenylate synthetase (OAS) which are also involved in antiviral defense (Parrish, S. et al., RNA. 7, 1397-402 (2001); Chen, C. C. et al., Curr Biol. 15, 378-83 (2005)). Thus, it is reasonable to speculate that the classical antiviral pathways that are activated by dsRNA recognition in mammalian cells may have evolved from the RNAi machinery. Whether this is true and whether the components of the classical antiviral pathways in mammalian cells also play a role in RNAi is unclear. Nevertheless, it is noteworthy that deletion of RIG-I in mice results in embryonic lethality (Kato, H. et al., Immunity. 23, 19-28 (2005)) similar to deletions of known components of the RNAi machinery such as Dicer and Argonaute 2 (Bernstein, E. et al, Nat Genet. 35, 215-7 (2003); Liu, J. et al., Science. 305, 1437-41 (2004)).

The ties between the RNAi pathway and the antiviral pathways in mammalian cells are stronger than initially believed (Chen, W., et al., FEBS Lett. 579, 2267-72 (2005)). RNAi appears to have retained its antiviral function in mammalian cells as in lower organisms although the action of intracellular dsRNA signaling pathways makes it difficult to pinpoint the specific contribution of each component. Nevertheless, the finding that dsRNAs are capable of triggering gene silencing despite the presence of 3′ overhangs suggests that recognition by RIG-I does not restrict their entry into the RNAi pathway. Therefore, it is possible that dsRNA signaling pathways and RNAi cooperate in antiviral defense in mammals.

TABLE
Summary of the dsRNAs used:

| | Duplex |
|---|---|
| target sequence (1)^{a} | size and structure |
| 5′gcaagctgaccctgaagttcatctgcaccaccggca 3′(SEQ ID NO: 7) | Synthesized |
| 5′GCUGACCCUGAAGUUCAUCUU (SEQ ID NO: 8) | 19 + 2 |
| 3′UUCGACUGGGACUUCAAGUAG (SEQ ID NO: 9) | |
| 5′AAGCUGACCCUGAAGUUCAUC (SEQ ID NO: 10) | 21 + 0 |
| 3′UUCGACUGGGACUUCAAGUAG (SEQ ID NO: 9) | |
| 5′AAGCUGACCCUGAAGUUCAUCUG (SEQ ID NO: 11) | 23 + 0 |
| 3′UUCGACUGGGACUUCAAGUAGAC (SEQ ID NO: 12) | |
| 5′GCUGACCCUGAAGUUCAUCUGUU (SEQ ID NO: 13) | 21 + 2 |
| 3′UUCGACUGGGACUUCAAGUAGAC (SEQ ID NO: 12) | |
| 5′AAGCUGACCCUGAAGUUCAUCUGCA (SEQ ID NO: 14) | 25 + 0 |
| 3′UUCGACUGGGACUUCAAGUAGACGU (SEQ ID NO: 15) | |
| 5′AAGCUGACCCUGAAGUUCAUCUGCACC (SEQ ID NO: 16) | 27 + 0 |
| 3′UUCGACUGGGACUUCAAGUAGACGUGG (SEQ ID NO: 17) | |
| 5′GCUGACCCUGAAGUUCAUCUGCACCACUU (SEQ ID NO: 18) | 27 + 2 |
| 3′UUCGACUGGGACUUCAAGUAGACGUGGUG (SEQ ID NO: 19) | |
| 5′aagcugacccugaaguucaucugcacc (SEQ ID NO: 16) | 27 + 0 DNA |
| 3′uucgacugggacuucaaguagacgugg (SEQ ID NO: 17) | |
| | Synthesized by IDT |
| 5′AAGCUGACCCUGAAGUUCAUCUGCACC (SEQ ID NO: 16) | 27 + 0 |
| 3′UUCGACUGGGACUUCAAGUAGACGUGG (SEQ ID NO: 17) | |
| 5′GCUGACCCUGAAGUUCAUCUGCACCAC (SEQ ID NO: 20) | 25 + 2 |
| 3′UUCGACUGGGACUUCAAGUAGACGUGG (SEQ ID NO: 17) | |
| 5′GCAAGCUGACCCUGAAGUUCAUCUGCA (SEQ ID NO: 21) | 25 − 2 |
| 3′UUCGACUGGGACUUCAAGUAGACGUGG (SEQ ID NO: 17) | |
| 5′GCAAGCUGACCCUGAAGUUCAUCUGCACCAC (SEQ ID NO: 22) | 25 +/− 2 |
| 3′UUCGACUGGGACUUCAAGUAGACGUGG (SEQ ID NO: 17) | |
| 5′AAGCUGACCCUGAAGUUCAUCUGCACC (SEQ ID NO: 15) | 25 + 2*(S) |
| 3′UUCGACUGGGACUUCAAGUAGACGU (SEQ ID NO: 16) | |
| 5′AAGCUGACCCUGAAGUUCAUCUGCA (SEQ ID NO: 14) | 25 − 2*(A) |
| 3′UUCGACUGGGACUUCAAGUAGACGUGG (SEQ ID NO: 17) | |
| 5′ACCCUGAAGUUCAUCUGCACCACcg (SEQ ID NO: 23) | 25 + 2*(A) + (S DNA end) |
| 3′ACUGGGACUUCAAGUAGACGUGGUGGC (SEQ ID NO: 24) | |
| 5′pACCCUGAAGUUCAUCUGCACCACcg (SEQ ID NO: 23) | 5′ P(S) − 25 + 2*(A) + |
| 3′ACUGGGACUUCAAGUAGACGUGGUGGC (SEQ ID NO: 24) | (S DNA end) |
| target sequence (2)^{a} | |
| 5′aagcagcacgacttcttcaagtccgcc 3′(SEQ ID NO: 25) | Synthesized |
| 5′AAGCAGCACGACUUCUUCAAGUCCGCC (SEQ ID NO: 26) | 27 + 0 (2) |
| 3′UUCGUCGUGCUGAAGAAGUUCAGGCGG (SEQ ID NO: 27) | |

^{a}Upper case letters represent RNA bases and lower case letters represent DNA

All references referred to herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of modulating activation of a double stranded RNA (dsRNA) signaling pathway that accompanies RNA interference (RNAi) of a target RNA sequence in a cell, comprising introducing into the cell small interfering RNA (siRNA) that degrades the target RNA sequence, and maintaining the cell under conditions in which RNAi of the target RNA sequence occurs and activation of the dsRNA signaling pathway is modulated in the cell.

2. The method of claim 1 wherein the dsRNA that accompanies the RNAi is induced.

3. The method of claim 2 wherein the siRNA that is introduced into the cell is double stranded and comprises at least one blunt end.

4. The method of claim 3 the siRNA is selected from the group consisting of:
a) an siRNA wherein both ends are blunt-ended;
b) an siRNA wherein one end is blunt-ended and the other end comprise a 2 nucleotide 5′ overhang;
c) an siRNA wherein one end is blunt-ended and the other end comprises a 2 nucleotide 3′ overhang; and
d) a combination thereof.

5. The method of claim 2 wherein the siRNA that is introduced into the cell is double stranded and comprise a 2 nucleotide 5′ overhang at each end.

6. The method of claim 1 wherein the dsRNA signaling pathway that accompanies the RNAi is inhibited.

7. The method of claim 6 wherein the siRNA that is introduced into the cell is double stranded and comprise at least 2 overhangs.

8. The method of claim 7 wherein the siRNA is selected from the group consisting of:
a) an siRNA wherein both 3′ ends comprise a 2 nucleotide overhang;
b) an siRNA wherein one end comprises a 3′ 2 nucleotide overhang and the other end comprises a 5′ 2 nucleotide overhang; and
c) a combination thereof.

9. The method of claim 1 wherein the siRNA comprises a sequence that is from about 19 to about 30 nucleotides.

10. The method of claim 9 wherein the siRNA comprises a sequence that is from about 25 to about 27 nucleotides.

11. The method of claim 1 wherein the siRNA comprises at least one overhang comprising from about 1 nucleotide to about 5 nucleotides.

12. The method of claim 11 wherein the overhang comprises 2 nucleotides.

13. A method of degrading a target RNA sequence using RNA interference (RNAi) in the presence of non-specific effects in a cell, comprising introducing into the cell small interfering RNA (siRNA) that degrades the target RNA sequence, and maintaining the cell under conditions in which the target RNA sequence is degraded by the siRNA in the presence of non-specific effects.

14. The method of claim 13 wherein the non-specific effects comprise the activation of double stranded RNA (dsRNA) signaling pathway and results in an inflammatory response, apoptosis or a combination thereof in the cell.

15. The method of claim 13 wherein the siRNA that is introduced into the cell is double stranded and comprises at least one blunt end.

16. The method of claim 15 the siRNA is selected from the group consisting of:
a) an siRNA wherein both ends are blunt-ended;
b) an siRNA wherein one end is blunt-ended and the other end comprise a 2 nucleotide 5′ overhang;
c) an siRNA wherein one end is blunt-ended and the other end comprises a 2 nucleotide 3′ overhang; and
d) a combination thereof.

17. The method of claim 13 wherein the siRNA that is introduced into the cell is double stranded and comprise a 2 nucleotide 5′ overhang at each end.

18. A method of degrading a target RNA sequence using RNA interference (RNAi) in the absence of non-specific effects in a cell, comprising introducing into the cell small interfering RNA (siRNA) that degrades the target RNA sequence, wherein the siRNA is double stranded and comprises at least 2 overhangs, and maintaining the cell under conditions in which the target RNA sequence is degraded by the siRNA in the absence of non-specific effects.

19. The method of claim 18 wherein the non-specific effect comprise activation of a double stranded RNA (dsRNA) signaling pathway which results in an inflammatory response, apoptosis in the cell or a combination thereof in the cell.

20. The method of claim 18 the siRNA is selected from the group consisting of:
a) an siRNA wherein both 3′ ends comprise a 2 nucleotide overhang;
b) an siRNA wherein one end comprises a 3′ 2 nucleotide overhang and the other end comprises a 5′ 2 nucleotide overhang; and
c) a combination thereof.

21. A method of enhancing an antiviral effect induced using RNA interference (RNAi) in a cell, comprising introducing into the cell small interfering RNA (siRNA) that degrades a target RNA sequence of a virus, wherein the siRNA promotes double stranded RNA (dsRNA) signaling in the cell, and maintaining the cell under conditions in which the siRNA degrades the target RNA sequence of the virus and promotes dsRNA signaling in the cell.

22. The method of claim 21 wherein the siRNA that is introduced into the cell is double stranded and comprises at least one blunt end.

23. The method of claim 22 the siRNA is selected from the group consisting of:
a) an siRNA wherein both ends are blunt-ended;
b) an siRNA wherein one end is blunt-ended and the other end comprise a 2 nucleotide 5′ overhang;
c) an siRNA wherein one end is blunt-ended and the other end comprises a 2 nucleotide 3′ overhang; and
d) a combination thereof.

24. The method of claim 21 wherein the siRNA that is introduced into the cell is double stranded and comprise a 2 nucleotide 5′ overhang at each end.

25. A method of enhancing an anticancer effect induced using RNA interference (RNAi) in a cell, comprising introducing into the cell small interfering RNA (siRNA) that degrades a target RNA sequence of a cancer, wherein the siRNA promotes double stranded RNA (dsRNA) signaling in the cell, and maintaining the cell under conditions in which siRNA degrades the target RNA sequence of the cancer and promotes dsRNA signaling in the cell.

26. The method of claim 25 wherein the siRNA that is introduced into the cell is double stranded and comprises at least one blunt end.

27. The method of claim 26 the siRNA is selected from the group consisting of:
a) an siRNA wherein both ends are blunt-ended;
b) an siRNA wherein one end is blunt-ended and the other end comprise a 2 nucleotide 5′ overhang;
c) an siRNA wherein one end is blunt-ended and the other end comprises a 2 nucleotide 3′ overhang; and
d) a combination thereof.

28. The method of claim 25 wherein the siRNA that is introduced into the cell is double stranded and comprise a 2 nucleotide 5′ overhang at each end.


SEQUENCE LISTING (27 sequences)

SEQ ID NO: 1 - DNA, 21 bp, Unknown
  primer for ISG56
gccacaaaaaatcacaagcca

SEQ ID NO: 2 - DNA, 21 bp, Unknown
  primer for ISG56
ccattgtctggatttaagcgg

SEQ ID NO: 3 - DNA, 20 bp, Unknown
  primer for human RIG-1
gactggacgtggcaaaacaa

SEQ ID NO: 4 - DNA, 26 bp, Unknown
  primer for RIG-1
ttgaatgcatccaatatacacttctg

SEQ ID NO: 5 - DNA, 18 bp, Unknown
  primer for human GAPDH
gaaggtgaaggtcggagt

SEQ ID NO: 6 - DNA, 20 bp, Unknown
  primer for human GAPDH
gaagatggtgatgggatttc

SEQ ID NO: 7 - DNA, 36 bp, Unknown
  GFP Target Sequence
gcaagctgaccctgaagttcatctgcaccaccggca

SEQ ID NO: 8 - RNA, 21 nt, Unknown
  sense strand of a dsRNA targeted against GFP
gcugacccugaaguucaucuu

SEQ ID NO: 9 - RNA, 21 nt, Unknown
  antisense strand of a dsRNA targeted against GFP
gaugaacuucagggucagcuu

SEQ ID NO: 10 - RNA, 21 nt, Unknown
  sense strand of a dsRNA targeted against GFP
aagcugacccugaaguucauc

SEQ ID NO: 11 - RNA, 23 nt, Unknown
  sense strand of a dsRNA targeted against GFP
aagcugacccugaaguucaucug

SEQ ID NO: 12 - RNA, 23 nt, Unknown
  antisense strand of a dsRNA targeted against GFP
cagaugaacuucagggucagcuu

SEQ ID NO: 13 - RNA, 23 nt, Unknown
  sense strand of a dsRNA targeted against GFP
gcugacccugaaguucaucuguu

SEQ ID NO: 14 - RNA, 25 nt, Unknown
  sense strand of a dsRNA targeted against GFP
aagcugacccugaaguucaucugca

SEQ ID NO: 15 - RNA, 25 nt, Unknown
  antisense strand of a dsRNA targeted against GFP
ugcagaugaacuucagggucagcuu

SEQ ID NO: 16 - RNA, 27 nt, Unknown
  sense strand of a dsRNA targeted against GFP
aagcugacccugaaguucaucugcacc

SEQ ID NO: 17 - RNA, 27 nt, Unknown
  antisense strand of a dsRNA targeted against GFP
ggugcagaugaacuucagggucagcuu

SEQ ID NO: 18 - RNA, 29 nt, Unknown
  sense strand of a dsRNA targeted against GFP
gcugacccugaaguucaucugcaccacuu

SEQ ID NO: 19 - RNA, 29 nt, Unknown
  antisense strand of a dsRNA targeted against GFP
guggugcagaugaacuucagggucagcuu

SEQ ID NO: 20 - RNA, 27 nt, Unknown
  sense strand of a dsRNA targeted against GFP
gcugacccugaaguucaucugcaccac

SEQ ID NO: 21 - RNA, 27 nt, Unknown
  sense strand of a dsRNA targeted against GFP
gcaagcugacccugaaguucaucugca

SEQ ID NO: 22 - RNA, 31 nt, Unknown
  sense strand of a dsRNA targeted against GFP
gcaagcugacccugaaguucaucugcaccac

SEQ ID NO: 23 - DNA, 25 bp, Unknown
  sense strand of a dsRNA targeted against GFP
acccugaaguucaucugcaccaccg

SEQ ID NO: 24 - RNA, 27 nt, Unknown
  antisense strand of a dsRNA targeted against GFP
cgguggugcagaugaacuucaggguca

SEQ ID NO: 25 - DNA, 27 bp, Unknown
  Target sequence for siRNA
aagcagcacgacttcttcaagtccgcc

SEQ ID NO: 26 - RNA, 27 nt, Unknown
  sense strand of a dsRNA targeted against GFP
aagcagcacgacuucuucaaguccgcc

SEQ ID NO: 27 - RNA, 27 nt, Unknown
  antisense strand of a dsRNA targeted against GFP
ggcggacuugaagaagucgugcugcuu